(19) United States
(12) Patent Application Publication
(10) Pub. No.: US 2024/0034831 A1
(43) Pub. Date: Feb. 1, 2024

(54) Process of preparing isocyanate compounds comprising non-chlorinated derivative and composition thereof

(71) Applicants: Keun Sik KIM, Suncheon-si, Jeollanam-do (KR); KS LABORATORIES CO., LTD., Suncheon-si, Jeollanam-do (KR)

(72) Inventor: Keun Sik KIM, Suncheon-si, Jeollanam-do (KR)

(21) Appl. No.: 18/026,840

(22) PCT Filed: Sep. 18, 2020
(86) PCT No.: PCT/KR2020/012629

§ 371 Date: Mar. 17, 2023

(51) Int. Cl. C08G 18/76; C07C 263/10; C07C 209/68; C07C 263/20; G02B 1/04

(52) U.S. Cl. CPC C08G 18/7642; C07C 263/10; C07C 209/68; C07C 263/20; G02B 1/041

(57) ABSTRACT

The present invention relates to a method for preparing an isocyanate containing a non-chlorinated derivative obtained after converting a chlorinated derivative, which is generated in a process for preparing xylylene diisocyanate, into the non-chlorinated derivative by means of a reduction reaction. In particular, the present invention relates to an isocyanate composition comprising the non-chlorinated derivative obtained by the reduction reaction and a polymerizable composition comprising the same.

### TECHNICAL FIELD

The present invention relates to a method for preparing an isocyanate by converting a chlorinated derivative, which is generated in a process for preparing xylylene diisocyanate, into a non-chlorinated derivative by means of a reduction reaction, and comprising it. In particular, the present invention relates to an isocyanate composition comprising a non-chlorinated derivative together with trace amounts of chlorinated derivatives, and a polymerizable composition comprising the same, wherein the chlorine derivatives, which are generated in a process of preparing xylylene diisocyanate, are converted into the non-chlorinated derivatives by means of a reduction reaction.

### BACKGROUND ART

Xylylene diisocyanate (hereinafter, abbreviated as “XDI”) is a compound that is very useful in the chemical, resin, and paint industries as a raw material for a polyurethane-based material, a polyurea-based material, and a polyisocyanate-based material. This may be divided into ortho-, meta-, or para-xylylene diisocyanate depending on the structural isomer, but among them, meta-xylylene diisocyanate (hereinafter, “m-XDI”; alternatively, also referred to as “1,3-bis (isocyanatometyl)benzene”) accounts for the majority. XDI is a specific diisocyanate having a methylene group in the benzene nucleus and is an isocyanate having characteristics of aromatic and aliphatic isocyanates. Since it has a property of preventing yellowing, which is a disadvantage of aromatic diisocyanate, it is used as a urethane raw material such as a non-yellowing-based paint, coating, leather, and adhesive.

Various methods for preparing XDI have been proposed in the prior art, but in general, various phosgenation methods have been developed. The phosgene method produces an isocyanate by reacting the reaction of an organic primary amine and phosgene in an inert solvent. In this method, the aromatic primary amine can relatively readily convert the high purity aromatic isocyanate by passing phosgene gas through the suspension of the free aromatic amine, carbonate or hydrochloride thereof in the solvent.

However, XDI, which contains an aromatic ring but is classified as an aliphatic isocyanate, generates a lot of side reactions during the reaction of the xylylene diamine and phosgene to prepare the isocyanate, and various chlorinated derivatives are generated as by-products.

It is stated that the chlorinated derivatives formed in the manufacture of conventional aliphatic isocyanates specified in Korean Patent Publication No. 1994-0001948 are generally formed in an amount of 3 to 10 wt %, and sometimes up to 20 wt %. Therefore, when the chlorinated derivative is contained in the XDI, the reaction between the isocyanate group and the active hydrogen-containing compound is affected when the polyurethane resin is manufactured from the XDI, thereby suppressing the reaction. In addition, it promotes the gelation of the initial polymer and seriously affects the quality of optical lenses such as yellowing, white turbidity, and striae.

Meanwhile, the phosgenation method may be divided into a direct method of directly reacting phosgene with a raw amine and a hydrochloride method of making the raw amine into a hydrochloride salt and then reacting it with phosgene. The direct method is much simpler than hydrochloride, but since significant amounts of chlorinated derivatives are produced, the direct method is not usually adopted. For this reason, in the case of preparing a chain-shaped or cyclic aliphatic isocyanate, the hydrochloride method is used in which isocyanate is produced by reacting raw amine with hydrochloride and then reacting with phosgene. However, since a certain amount of chlorinated derivatives are produced in the hydrochloride method, methods for obtaining high-purity XDI by reducing the amount of impurities produced have been additionally disclosed.

Korean Patent No. 10-0953019 discloses method for preparing an isocyanate from chain or cyclic aliphatic amine, in which a high pressure is applied during a crude salt reaction process (a process of obtaining a slurry containing amine hydrochloride) to suppress an increase in the diameter of the hydrochloride. In addition, it is disclosed that the production of yield and chlorinated derivatives can be reduced to 0.1-0.3 wt % by increasing the conversion rate of hydrochloride when phosgenating by improving flowability and binucleate property through the reduction of hydrochloride viscosity by miniaturization of hydrochloride particles.

Furthermore, Korean Patent Laid-Open Publication No. 2018-0104330 discloses that a resin for an optical material obtained in an XDI composition in which the concentration of the compound of Chemical Formula (5), as a chlorinated derivative, is 0.2 ppm to less than 600 ppm, has excellent sulfur resistance and high production efficiency. In addition, Korean Patent Laid-Open Publication No. 2018-0127517 discloses that the compound of chemical Formula (7) among chlorinated derivatives should be present at 60 ppm or less to have excellent discoloration resistance. However, chlorinated derivatives generated as major impurities in the isocyanate synthesis reaction serve as impurities that adversely affect the physical properties of the optical lens in the application of the polyurethane reaction. It is determined that the content of these impurities causes yellowing and does not have a good effect on the product, and thus these chlorinated derivatives may be substances that should not be produced or must be removed by purification even if they are produced. In all of these prior arts, since there is a problem in that precise control and separation and purification using a high-stage distillation column are required in the process, it is judged that economic feasibility is deteriorated due to an increase in device cost and commercialization possibility is low.

### PRIOR ART DOCUMENT

### Patent Document

(Patent Document 1) Korean Patent Publication No. 1994-0001948 (Registered on Mar. 12, 1994)
(Patent Document 2) Korean Patent No. 0953019 (Registration on Apr. 7, 2010)
(Patent Document 3) Korean Patent Laid-Open Publication No. 2018-0104330 (Published on Sep. 20, 2018)
(Patent Document 4) Korean Patent Laid-Open Publication No. 2018-0127517 (Published on Nov. 28, 2018)

### DETAILED DESCRIPTION OF THE INVENTION

### Technical Problem

To this end, the present invention is intended to produce isocyanate by simplifying the process by converting the chlorinated derivative produced in a general process into a non-chlorinated derivative through a simple reduction reaction without proceeding with the above complicated process for reducing the chlorinated derivative.

Here, the non-chlorinated derivative formed by the reduction reaction has excellent sulfur resistance even if the non-chlorinated derivative is included in some resins, improves physical properties of a general resin, and has a boiling point lower than that of XDI by 60 to 75° C., thereby simplifying separation and purification. In general, in order to suppress the production of chlorinated derivatives, hydrochlorination of amines, high-pressure reaction in the crude salt production process, and separation and purification through precise control are required. However, in the present invention, even in a direct method process in which it is difficult to control a chlorinated derivative, separation and purification may be simplified by a simple reduction reaction to have economic advantages, thereby being differentiated from conventional processes. Therefore, according to the present invention, it is possible to bring about economic benefits in the industrial field by reducing process cost, obtaining high yield and high quality products.

### Technical Solution

To achieve the object of the present invention, in one aspect, the present invention provides method for preparing an isocyanate, comprising the non-chlorinated derivative obtained after converting a chlorinated derivative represented by chemical Formula (1), generated in a process for preparing xylylene diisocyanate, into a non-chlorinated derivative by means of a reduction reaction:

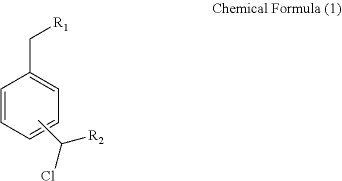

(wherein R₁ is Cl or NCO, and R₂ is H or Cl.)

The xylylene diisocyanate of the present invention may be obtained by reacting an organic primary amine directly with a carbonylating agent in an inert solvent according to the following schemes:

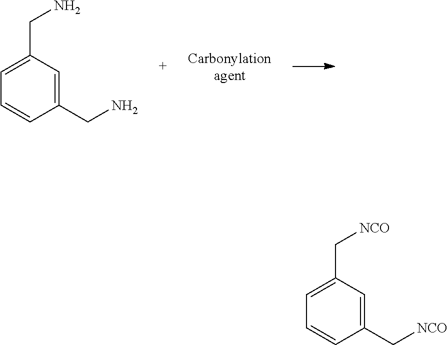

Also, the xylylene diisocyanate of the present invention may be obtained by a method comprising a first step of reacting an organic primary amine with hydrogen chloride gas, and a second step of reacting the amine hydrochloride produced in the first step with a carbonylating agent, according to the following reaction scheme:

Step 1

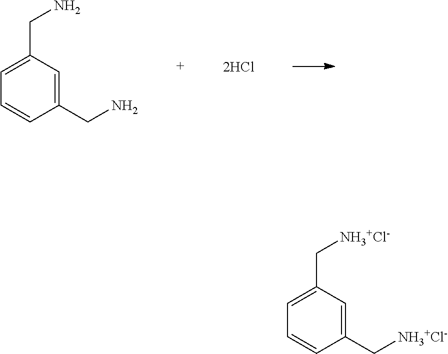

Step 2

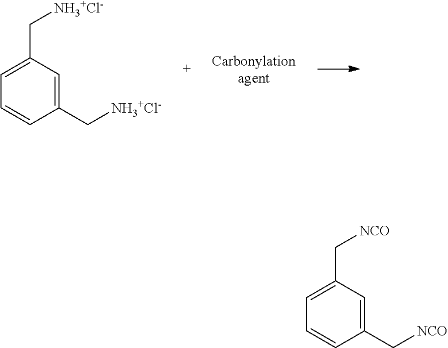

The carbonylating agent used in the present invention may be selected from phosgene, diphosgene, triphosgene, alkylchloroformate, or a combination thereof. In addition, the chlorinated derivative may be at least one of the chlorinated derivatives represented by the following Chemical Formula (2):

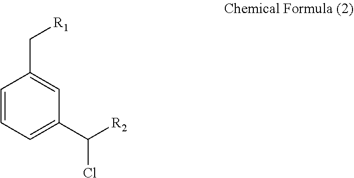

(R₁ is Cl or NCO, and R₂ is H or Cl.)

Furthermore, the non-chlorinated derivative may be at least one of the non-chlorinated derivatives represented by the following Chemical Formula (3):

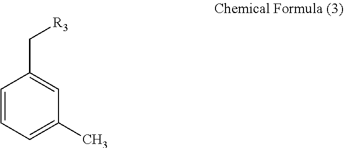

(R₃ is H or NCO.)

Meanwhile, the reduction reaction of the present invention may be performed using a metal catalyst or a reducing agent under a hydrogen (H2) atmosphere, and the metal catalyst used herein may be at least one of palladium, platinum, and nickel, and palladium or platinum is preferable. In addition, the reducing agent used may be at least one selected from the following:

Lithium aluminum hydride (LiAlH4), sodium amalgam (Na(Hg)), zinc amalgam (Zn(Hg)), diborane (B2H6), lithium boro hydride (LiBH4), sodium boro hydride (NaBH4), iron (II) sulfate (FeSO4), tin (II) chloride (SnCl2), sodium dithionate (Na2S2O6), sodium thiosulfate (Na2S2O3), ammonium thiosulfate ((NH4)2S2O3), diisobutylaluminum hydride (DIBAL-H), oxalic acid (C2H2O4), formic acid (HCOOH), dithiotretol (DTT), tris-2-carboxyethyl fospinhydrochloride (TCEP), 2,2-diphenyl-1-picrylhydrazyl (DPPH), butylated hydroxytoluene (BHT), 2,6-di-tert-butyl-4-methylphenol, and 2,4-dimethyl-6-tert-butylphenol, metallic hydrides such as trialkyltin hydride or tributyltin hydride, metal such as zinc.

In another aspect, the present invention provides an isocyanate composition comprising xylylene diisocyanate (XDI) as a main component, wherein the isocyanate composition further comprises methylbenzyl isocyanate represented by Formula (4) as a non-chlorinated derivative obtained by the above preparation method:

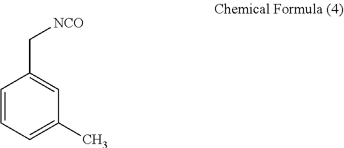

In addition, the isocyanate composition of the present invention may further include a chlorinated derivative represented by the above-described Chemical Formula (1). In addition, the chlorinated derivative is a chlorinated derivative represented by Chemical Formula (2), and may further include at least one of chloromethylbenzyl isocyanate, bis(chloromethyl) benzene, dichloromethylbenzyl isocyanate, and chloromethyl dichloromethyl benzene:

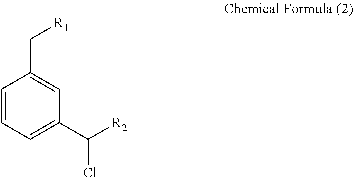

(R₁ is Cl or NCO, and R₂ is H or Cl.)

In addition, the isocyanate composition of the present invention may contain a non-chlorinated derivative in a range of 0.1 to 50, 000 ppm, and may contain a chlorinated derivative in a range of greater than 0 to 1, 500 ppm. Furthermore, in the isocyanate composition of the present invention, when the isocyanate is xylylene diisocyanate (XDI), it is preferable to contain 99% by mass or more in consideration of various properties of the optical lens.

Further, in order to achieve a further object of the present invention, in yet another aspect, the present invention provides a polymerizable composition comprising the isocyanate composition and a compound comprising at least one isocyanate-reactive functional group. Here, the compound containing at least one isocyanate-reactive functional group used may be a polyol compound or a polythiol compound, and the polymerizable composition may be used as a coating material.

Furthermore, in order to achieve an additional object of the present invention, as another aspect, the present invention provides a resin obtained by reacting the above-described polymerizable composition. The resin may be used as an optical material containing an optical lens.

Meanwhile, as another aspect, the present invention may provide an isocyanate composition further comprising, as a main component, xylylene diisocyanate further comprising methylbenzyl isocyanate represented by the Chemical Formula (4), as a non-chlorinated derivative obtained by separately synthesizing the chlorinated derivative, not obtained by a reduction reaction.

As described above, the isocyanate composition may further include one or more of the chlorinated derivatives represented by Chemical Formula 2 as the chlorinated derivatives. Here again, the isocyanate composition preferably contains a non-chlorinated derivative in the range of 0.1 ppm to 50,000 ppm, and preferably contains a non-chlorinated derivative in the range of greater than 0 to 1,500 ppm.

### Effects of Invention

The preparation method of the present invention can improve preparation efficiency and quality of an isocyanate which is widely used in chemical, resin, and paint industries containing the field of optical materials, thereby has high technical and industrial value.

The isocyanate composition comprising the non-chlorinated derivative obtained through the reduction process of the present invention has excellent economic efficiency and can easily reduce the chlorinated derivative. Also, by using it, the optical resin or optical product, which has excellent quality, can be obtained with high economic efficiency.

### BRIEF DESCRIPTION OF DRAWING

FIG. 1 is a graph showing the results of gas chromatography (GC) of the product of Example 3 before the reduction reaction (A) and after the reduction reaction (B) of Preparation Example 3 and before the desolvation process, in the process of converting the chlorinated derivative into a non-chlorinated derivative through a simple reduction reaction.

### BEST MODE FOR CARRYING OUT THE INVENTION

### Definition

Unless otherwise defined, all technical and scientific terms used in the description of the present invention have the same meaning as those generally understood by those skilled in the art belonging to the present invention. All patent publications, application publications, and other publications cited as prior art are incorporated as reference.

As used in the description of the present invention, “isocyanate” is a material used for polyurethane-based materials and polyurea-based materials, and is used to synthesize these materials having different structures depending on the number and location of functional groups. Here, “isocyanate” is used as the meaning containing all of monoisocyanate, diisocyanate, and polyisocyanate.

The term “chlorinated derivative” occurs when isocyanate is produced by the direct method, the hydrochloride method or the carbonate method, and refers to impurities containing chlorine generated by side reactions. In the present invention, chlorinated impurities include 3-chloromethylbenzyl isocyanate (Formula 5), 1,3-xylylene dichloride (Formula 6), 3-dichloromethylbenzyl isocyanate (Formula 7), and the like:

Chemical Formula (5); Chemical Formula (6); Chemical Formula (7)

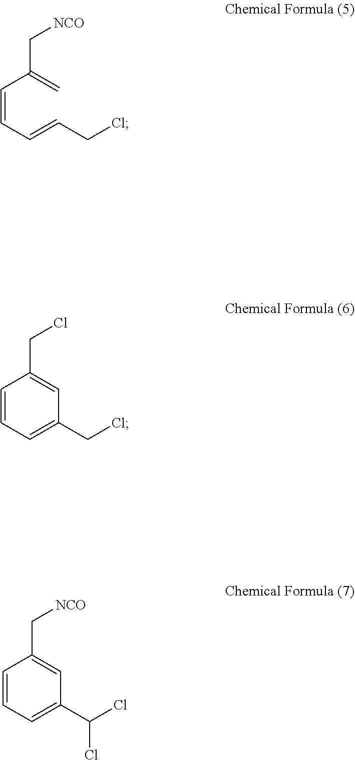

The term “non-chlorinated derivative” refers to a substance in which chlorine is replaced with other atoms or atoms to remove it, by a reduction reaction for the “chlorinated derivative”. In the present invention, the non-chlorinated impurities include methylbenzyl isocyanate, xylene, and the like.

In addition, the term “isocyanate-reactive functional group” refers to a “active hydrogen group-containing component” that reacts with an isocyanate, for example, a polyol component (a component mainly contained in a polyol having two or more hydroxyl groups), a polythiol component (a component mainly contained in a polythiol having two or more mercapto groups), a polyamine (a component mainly contained in a polyamine having two or more amino groups), and the like.

The term “carbonylation agents” refers to reagents that cause a carbonylation reaction, which is a reaction of introducing carbon monoxide into organic compounds. Carbonylation results in compounds containing organic carbonyl, i.e., >C═O functional groups such as aldehydes, ketones, carboxylic acids and esters. In particular, since organic compounds containing a carbonyl group have an unsaturated bond, they are highly reactive and can be highly selective, so they are widely used in synthetic chemistry. Representative carbonylating agents used in the present invention include phosgene, diphosgene, triphosgene, chloroformate-based compounds, and the like.

As used herein, the term “combination” includes blends, mixtures, reaction products, and the like.

In addition, specific values of a mixing ratio (content ratio), a property value, a parameter, and the like described in the present invention may be replaced by an upper limit (a value defined as “less than or equal” or “less”) or a lower limit (a value defined as “more than or equal” or “more”) of a corresponding description such as a mixing ratio (content ratio), a property value, a parameter, and the like corresponding thereto. On the other hand, “%” is based on mass unless otherwise specified.

The singular form, as used in the description and claims of the present invention, includes a plurality of objects to be indicated unless the context clearly indicates otherwise. Thus, for example, reference to “isocyanate” includes mixtures of two or more monoisocyanates, diisocyanate, and polyisocyanate.

Each of the materials disclosed in the description of the present invention is commercially available and methods of production thereof are known to those skilled in the art, unless specifically stated. In addition, all test standards are the most recent standards effective at the time of this application unless otherwise stated in the description of the present invention.

Hereinafter, a method and a composition for preparing the isocyanate of the present invention will be described in detail.

First, the present invention aims to easily increase the purity of isocyanate without going through a complicated high-stage purification process by converting a chlorinated derivative produced in the process of producing isocyanate into a non-chlorinated derivative through a reduction reaction of the following reaction equation:

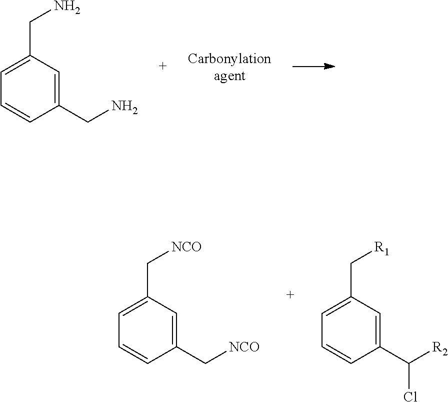

(R₁ is Cl or NCO, and R₂ is H or Cl.)

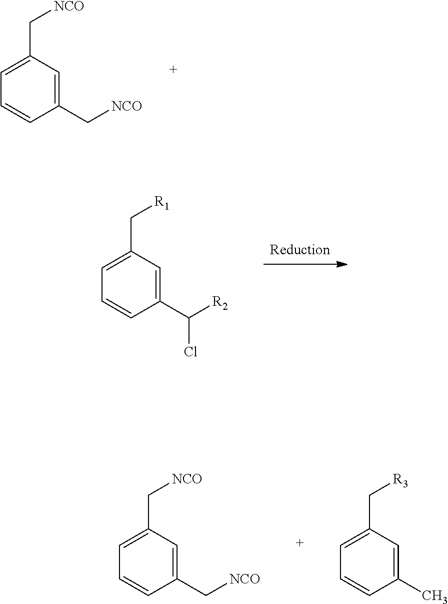

(R₃ is H or NCO.)

As described above, in the hydrochloride method, as in the direct method, a predetermined amount of chlorinated derivatives known as the compound of Chemical Formula 5 are produced, and thus, they may be used to obtain XDI constituting the isocyanate composition of the present invention.

The XDI includes structural isomers 1,2-XDI (ortho-form), 1,3-XDI (meta-form), and 1,4-XDI (para-form), which may be used alone or in combination with two or more, preferably 1,3-XDI or 1,4-XDI, more preferably 1,3-XDI.

Since the isocyanate composition obtained by the preparation method of the present invention is used as a reactant in order to obtain a polymer compound such as polyurethane, the aliphatic amine used in the present invention is preferably a chain or cyclic aliphatic amine having at least two functional properties.

Preferably used in the present invention, the two functional or more chain or cyclic aliphatic amines are not particularly limited, but will be referred to in Patent No. 10-0953019, which is the prior document described above. Representative examples thereof include chain aliphatic amines such as hexamethylenediamine, 2,2-dimethylpentanediamine, 2,2,4-trimethylhexanediamine, butenediamine, xylylenediamine, and cyclic aliphatic amines such as bis(aminomethyl)cyclohexane, dicyclohexylmethanediamine, cyclohexanediamine, and bis(aminomethyl)norbornene.

The isocyanate obtained by reacting the above-described chain or cyclic aliphatic diamine with phosgene will be determined according to the reacting diamine, and will be referred to in Patent No. 10-0953019, which is a prior document. Representative examples thereof may include chain aliphatic isocyanates such as hexamethylene diisocyanate, 2,2-dimethylpentane diisocyanate, 2,2,4-trimethylhexane diisocyanate, butene diisocyanate, xylylene diisocyanate, and the like, cyclic aliphatic isocyanates such as bis(isocyanatomethyl) cyclohexane, dicyclohexylmethane diisocyanate, cyclohexane diisocyanate, bis(isocyanatomethyl) norbornene, and the like. Among the exemplary compounds obtained by the preparation method of the present invention, a particularly preferred compound for various optical device applications may include xylylene diisocyanate, bis(isocyanatomethyl) norbornene, hexamethylene diisocyanate, and bis(isocyanatomethyl) cyclohexane.

Carbonylation agents that can be used in the production of XDI of the present invention are phosgene, diphosgene, triphosgene and various chloroformate-based compounds, which can be used alone or in combination of two kinds.

Phosgene gas, which can be used in the preparation of xylylene diisocyanate of the present invention, is a commonly used name of carbonyl chloride or carbonyl dichloride (COCl₂), is a colorless gas at room temperature, having a solidification point of 127.84° C. and a boiling point of 7.84° C. It is generally produced by using a catalytic reaction of high purity carbon monoxide from an anhydrous chlorine gas. Phosgene substitutes and/or precursors used in accordance with the present invention may include any phosgene equivalents such as diphosgene, triphosgene, and the like, and any combinations thereof. The phosgene used in the present invention may be provided by pyrolysis of a carbamic acid derivative using chloroform, diphenyl carbamate, N, N′-carbonyldiimidazole, or the like.

Meanwhile, the isocyanate composition according to the present invention may include a small amount of a non-chlorinated derivative, wherein the non-chlorinated derivative is obtained from a chlorinated derivative represented by the chemical Formula (1) generated in the process of preparing XDI, through a reduction reaction using a catalyst or a reducing agent to convert the chlorinated derivative into a non-chlorinated derivative, and then through filtration and purification:

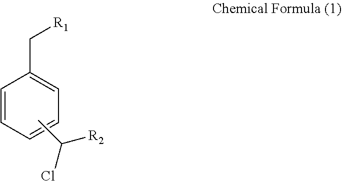

(wherein R₁ is Cl or NCO, and R₂ is H or Cl).

Here, the chlorinated derivative included in the XDI may include 3-chloromethylbenzyl isocyanate (Chemical Formula 5), 1,3-xylylene dichloride (Chemical Formula 6), 3-dichloromethylbenzyl isocyanate (Chemical Formula 7), and the like, and may be present alone or in two or more kinds.

Meanwhile, the non-chlorinated derivative will be methylbenzyl isocyanate (chemical Formula (4)) or xylene or the like. The compound of Formula (4) of the present invention comprises structural isomers 1,2-methylbenzyl isocyanate, 1,3-methylbenzyl isocyanate, 1,4-methylbenzyl isocyanate, like the structural isomers of XDI, and can be present alone or in two or more types, preferably 1,3-methylbenzyl isocyanate, 1,4-methylbenzyl isocyanate, more preferably 1,3-methylbenzyl isocyanate. Also, in the case of xylene, it includes structural isomers 1,2-xylene, 1,3-xylene, and 1,4-xylene, and may be present alone or in two or more types, preferably 1,3-xylene, 1,4-xylene, and more preferably 1,3-xylene.

In the XDI composition of the present invention, methylbenzyl isocyanate may be contained. When the amount of the methylbenzyl isocyanate is greater than 50, 000 ppm with respect to the total mass of the XDI composition, physical properties as a lens of the methylbenzyl isocyanate such as heat resistance are lowered, and then the methylbenzyl isocyanate is preferably 0.1 ppm or more to 50, 000 ppm or less. In addition, trace amounts of the chlorinated derivative can remain after conversion into methylbenzyl isocyanate. When the chlorinated derivative exceeds 1500 ppm in the presence of methylbenzyl isocyanate, the yellow index of the lens is increased, and thus the content of the chlorinated derivative preferably ranges from greater than 0 to less than or equal to 1,500 ppm with respect to the total mass of the XDI composition.

Meanwhile, the reduction reaction of the present invention is a dechlorine reaction by hydrogenation, and the reaction may be divided into a reduction process using a metal catalyst under a hydrogen atmosphere and a reduction process using a reducing agent, and may be performed alone or in parallel. Preferably, it is a reduction process using a noble metal catalyst in a hydrogen atmosphere.

The temperature of the reduction process is not particularly limited, but may be performed at 0° C. to 180° C., preferably 10 to 150° C., more preferably 20 to 50° C. Furthermore, the hydrogen pressure of the reduction process is not particularly limited, but may be performed at 0.001 kgf/cm² to 200 kgf/cm², 0.1 to 10 kgf/cm², and more preferably 1 to 5 kgf/cm².

In addition, the catalyst used in the reduction process is not particularly limited, but a noble metal and a transition metal catalyst may be used, and may be used alone or in combination with two or more kinds. The metal catalyst may be used alone as Pd, Pt, Ru, Ni, or the like or by supporting on a metal oxide such as activated carbon, SiO2, Al2O3, CeO2, ZrO2, or the like, and may be used alone or in combination of two or more kinds of metal ligands, or the like. In the reduction process, the amount of catalyst is not particularly limited, but may be 0.1% or more to 20% or less, preferably 0.3 to 15%, and more preferably 0.5 to 5% based on the total XDI mass. In addition, the reducing agent may be one or more kinds selected from the group consisting of lithium aluminum hydride (LiAlH4), sodium amalgam (Na(Hg)), zinc amalgam (Zn(Hg)), diborane (B2H6), lithium borohydride (LiBH4), sodium borohydride (NaBH4), iron (II) sulfate (FeSO4), tin (11) chloride (SnCl2), sodium dithionate (Na2S2O6), sodium thiosulfate (Na2S2O3), ammonium thiosulfate ((NH4)2S2O3), diisobutylaluminum hydride (DIBAL-H), oxalic acid (C2H2O4), formic acid (HCOOH), dithiothitol (DTT), 2,2-diphenyl-1-picrylhydrazyl (DPPH), butylated hydroxytoluene (BHT), 2,6-di-tert-butyl-4-methylphenol, and 2,4-dimethyl-6-tert-butylphenol, metallic hydride such as trialkyltin hydride or tributyltin hydride, and metal such as zinc. Preferably, a Pd/C catalyst is mentioned.

The reduction process of the present invention is preferably carried out in a nitrogen atmosphere or a hydrogen atmosphere in order to exclude oxygen, and the chlorinated derivative may be carried out in a solvent or may be carried out in a state in which the solvent is removed. The solvent used in the present invention is also not particularly limited. As an inert solvent, aromatic hydrocarbons such as benzene, toluene, xylene, etc., for example, aliphatic hydrocarbons such as octane, decane, etc., for example, cycloaliphatic hydrocarbons such as cyclohexane, methylcyclohexane, ethylcyclohexane, etc., for example, halogenated aromatic hydrocarbons such as chlorotoluene, chlorobenzene, dichlorobenzene, dibromobenzene, trichlorobenzene, etc., for example, nitrogen-containing compounds such as nitrobenzene, N,N-dimethylformamide, N,N-dimethylacetates amide, N,N′-dimethylimidazolidinone, etc., for example, ethers such as dibutyl ether, ethylene glycol dimethyl ether, ethylene glycol diethyl ether, etc., for example, fatty acid esters such as amyl formate, n-butyl acetate, isobutyl acetate, n-amyl acetate, isoamyl acetate, methylisomyl acetate, methoxyethyl acetate, 2-ethoxyethyl acetate, sec-hexyl acetate, 2-ethylbutyl acetate, 2-ethylhexyl acetate, cyclohexyl acetate, methylcyclohexyl acetate, benzyl acetate, ethyl propionate, n-butyl propionate, isoamyl propionate, ethyl acetate, butyl stearate, butyl lactate and amyl lactate, and aromatic carboxylic acid esters such as methyl salicylate, dimethyl phthalate, and methyl benzoate can be mentioned. The solvent may be used alone or in combination of two or more. Preferably halogenated aromatic hydrocarbons are mentioned, and more preferably chlorobenzene and dichlorobenzene are mentioned.

In addition, after filtering a catalyst or a reducing agent used through the reduction step of the present invention, the amount of the chlorinated and non-chlorinated derivatives can be controlled through simple distillation purification or the like. Of course, the reduction process may be applicable after preparing the isocyanate containing the chlorinated derivative together with XDI, after desolvation process, and after purification process.

Therefore, a product manufactured by using the isocyanate composition may satisfy high optical properties, and thus may be used for preparing an optical material, specifically, a plastic optical lens.

According to the present invention, there is provided a polymerizable composition comprising the isocyanate composition described above and a polyol/polythiol.

The polymerizable composition may comprise the isocyanate composition and the polyol/polythiol in a mixed state or in a separated state. That is, in the polymerizable composition, the isocyanate composition and the polyol/polythiol may be in a state of being mixed in contact with each other or may be in a state of being separated so as not to contact each other.

As a polyol component used in the polymerizable composition of the present invention, for example, low molecular weight polyol and high molecular weight polyol may be mentioned. The polyol may be used alone or in a combination of two or more.

The low molecular weight polyol is a compound with a number average molecular weight of 60 or more to less than 400 with two or more hydroxyl groups. As the low molecular weight polyols, the following may be exemplified: ethylene glycol, propylene glycol, 1,3-propenediol, 1,4-butylene glycol, 1,3-butylene glycol, 1,2-butylene glycol, 1,5-pentenediol, 1,6-hexenediol, diethylene glycol, triethylene glycol, dipropylene glycol, and mixtures thereof, dihydric alcohols such as 1,4-cyclohexenediol, hydrogenated bisphenol A, bisphenol A, and the like, for example, trihydric alcohols such as glycerin, and the like, for example, tetrahydric alcohol such as tetramethylolethane (pentaerythritol), and the like, for example, pentahydric alcohols such as xylitol, and the like, for example, hexahydric alcohols such as sorbitol, mannitol, allitol, iditol, and the like.

The high molecular weight polyol is a compound having a number average molecular weight of 400 or more, for example, 10000 or less, and preferably 5000 or less, with two or more hydroxyl groups. As the high molecular weight polyols, the following may be exemplified: polyether polyols, polyester polyols, polycarbonate polyolethane, polyurethane polyols, epoxy polyols, vegetable oil polyols, polyolefin polyols, acrylic polyols, silicon polyols, fluorine polyols, and vinyl monomer modified polyols.

Examples of the polythiol component used in the polymerizable composition of the present invention include aliphatic polythiol, aromatic polythiol, heterocyclic-containing polythiol, aliphatic polythiol containing a sulfur atom other than a mercapto group, aromatic polythiol containing a sulfur atom other than a mercapto group, and heterocyclic polythiol containing a sulfur atom other than a mercapto group. The thiol may be thiol oligomer or polythiol, and may be used alone or in a combination of two or more. Specific examples of the thiol include 3,3′-thiobis-[(2-mercaptoethyl)thio]-1-propanethiol, bis(2-(2-mercaptoethylthio)-3-mercaptopropyl)sulfide, 4-mercaptomethyl-1,8-dimercaptomethyl-3,6-dithiooctane, 2,3-bis(2-mercaptoethylthio)propan-1-thiol, 2,2-bis(mercaptomethyl)-1,3-propanedithiol, bis(2-mercaptoethyl)sulfide, tetrakis(mercaptomethyl)methane, 2-(2-mercaptoethylthio)propan-1,3-dithiol, 2-(2,3-bis(2-mercaptoethylthio)propylthio)ethanethiol, bis(2,3-mercaptoethanediyl)sulfide, bis(2,3-dimercaptoethylthio)disulfide, 1,2-bis[(2-mercaptoethyl)thio]-3-mercaptopropane, 1,2-bis(2-(2-mercaptoethylthio)-3-mercaptopropylthio)ethane, 2-(2-mercaptoethylthio)-3-2-mercapto-3-[3-mercapto-2-(2-mercaptoethylthio)-propylthio]propylthio-propane-1-thiol, 2,2-bis-(3-mercaptopropionyloxymethyl)butylester, 2-(2-mercaptoethylthio)-3-(2-[3-mercaptoethylthio)-propylthio]ethylthio)propan-1-thiol, (4R,11S)-4,11-bis(mercaptomethyl)-3,6,9,12-tetrathiatetradecan-1,14-dithiol, (S)-3-((R-2,3-mercaptopropyl)thio)propan-1,2-dithiol, (4R,14R)-4,14-bis(mercaptomethyl)-3,6,9,12,15-pentathiaheptane-1,17-dithiol, (S)-3-((R-3-mercapto-2-((2-mercaptoethyl)thio)propyl)thio)-2-((2-mercaptoethyl)thio)propane-1-thiol, 3,3′-dithiobis(propan-1,2-dithiol), (7R,11 S)-7,11-bis(mercaptomethyl)-3,6,9,12,15-pentathiaheptadecane-1,17-dithiol, (7R,12S)-7,12-bis(mercaptomethyl)-3,6,9,10,13,16-hexaoctadecane-1,18-dithiol, 5,7-dimercaptomethyl-1,11-dimercapto-3,6,9-trithianeundecane, 4,7-dimercaptomethyl-1,11-dimercapto-3,6,9-trithiaundecane, 4,8-dimercaptomethyl-1,11-dimercapto-3,6,9-trithiaundecane, pentaerythritol tetrakis(3-mercaptopropionate), trimethylolpropane tris(3-mercaptopropionate), pentaerythritol tetrakis(2-mercaptoacetate), bis pentaerythritol etherhexakis(3-mercaptopropionate), 1,1,3,3-tetrakis(mercaptomethylthio)propane, 1,1,2,2-tetrakis(mercaptomethylthio)ethane, 4,6-bis(mercaptomethylthio)-1,3-dithiane, pentaerythritol tetrakis(2-mercaptoacetate), pentaerythritol tetrakis(3-mercaptopropionate), 2-(2,2-bis(mercaptodimethylthio)ethyl)-1,3-dithiane, and the like.

In addition, the polymerizable composition may further include additives such as an internal release agent, an ultraviolet absorber, a near-infrared absorber, a polymerization initiator, a heat stabilizer, a color correction agent, a chain extender, a crosslinking agent, a light stabilizer, an antioxidant, a filler, and the like, if necessary.

As the internal release agent, components may be used alone or in combination of two or more thereof, selected from the following: a fluorine-based nonionic surfactant having a perfluoroalkyl group, a hydroxyalkyl group, or a phosphate ester group; silicon-based nonionic surfactant having a dimethylpolysiloxane group, a hydroxyalkyl group, or a phosphate ester group; quaternary ammonium salts of the alkyl group, i.e., trimethylcetyl ammonium salt, trimethylstearyl, dimethylethylcetyl ammonium salt, triethyldodecyl ammonium salt, trioctylmethyl ammonium salt, diethylcyclohexadiadodecyl ammonium salt; the acidic phosphoric esters.

The UV absorber may be benzophenone-based, benzotriazole-based, triazine-based, salicylate-based, cyanoacrylate-based, oxanilide-based, or the like.

Examples of the near-infrared absorber may include azo-based, aminium-based, anthraquinone-based, cyanine-based, polymethine-based, diphenylmethane-based, triphenylmethane-based, quinone-based, diimonium-based, dithiol-based metal complex-based, scuaryllium-based, phthalocyanine-based, and naphthalene-ocyanine-based, or the like. Particularly, as one of the electromagnetic wave absorbents, a near-infrared absorber having a high near-infrared absorption capacity of 30% or more of blocking rate in about 800 to 1000 nm, may be used. The near-infrared absorber is a mixture of a plurality of phthalocyanine-based pigments having different structures, and it is preferable that the pigments are pigments having a minimum value of a spectral transmittance curve of less than 80%, respectively, within a range of (i) a wavelength range of 800 nm to 850 nm, (ii) a wavelength range of 875 nm to 925 nm, and (iii) a wavelength range of 950 nm to 1000 nm. For example, PANAX FND-83, PANAX FND-88, PANAX FND-96, or the like may be used.

The polymerization initiator may be amine-based, phosphorus-based, organotin-based, organocopper-based, organogallium, organoorganozirconium, organoiron-based, organozinc, organoaluminum, or organobismuth-based.

As the heat stabilizer, metal fatty acid salt-based, phosphorus-based, lead-based, organotin-based, or the like may be used alone or in a combination of two or more thereof.

According to the present invention, polythiourethane obtained from the above-described polymerizable composition is provided. That is, the polythiourethane may be prepared by polymerizing (and curing) the isocyanate composition and thiol in the polymerizable composition. The polymerization reaction may be carried out such that the molar ratio of the SH group/NCO group is 0.5 to 3.0, more specifically 0.8 to 1.3.

In addition, in order to control the reaction rate, a reaction catalyst commonly used in the preparation of polythiourethane may be added. As the curing catalyst (polymerization initiator), a tin-based catalyst may be used, for example, dibutyltin dichloride, dibutyltin dilaurate, dimethyltin dichloride, or the like.

Afterwards, a method of measuring physical properties containing the specification and content of standard materials of various isocyanates should be described.

[Analysis Method]

### (1) Content of 1,3-xylene diisocyanate (XDI)

The XDI of 99% purity prepared in the preparation examples described below was analyzed by gas chromatography, as a standard material, under the following conditions, and a calibration curve was prepared from the obtained area of the gas chromatogram and quantified.
Device; HP-6890 (HP)
Column; DB-1, (inner diameter 0.53 mm×length 60 m×thickness 1.5 um)
Inlet temperature; 180° C.
Detector temperature; 300° C.

### (2) Content of 3-chloromethylbenzyl isocyanate (Chemical Formula (5))

A compound of Formula (5) having a purity of 99% separated/purified through column distillation in the XDI composition prepared in Preparation Example was used as a standard material, and the content of the compound of Formula (5) in the compositions of each Example and Comparative Example was calculated in the same manner as the measurement method of XDI.

The obtained compound of Formula (5) was analyzed by ¹³C-NMR (100 MHz, CDCl₃), FT-IR, MS.

¹³C-NMR (100 MHz, CDCl₃) δ 46.5, 54.6, 125.6, 127.9, 128.8, 130.5, 139.9, 139.1

FT-IR: 2260 cm⁻¹

MS: m/z=181(M′)

### (3) Content of 1,3-xylylene dichloride (Chemical Formula (6))

A compound (6) having a purity of 98% (reagent, Sigma-aldrich) was used as a standard material, and the content of the compound (6) in the compositions of each Example and Comparative Example was calculated in the same manner as the measurement method of XDI.

### (4) Content of dichloromethylbenzene isocyanate (Chemical Formula (7))

A compound of Formula (7) having a purity of 99 mol % prepared by the synthesis method specified in Korean Patent Laid-Open Publication No. 2018-0127517 was analyzed by gas chromatography, as a standard material, under the following conditions, and a calibration curve was prepared from the area of the obtained gas chromatogram and quantified.
Device; HP-6890 (HP)
Column; HP-50+, (inner diameter 0.25 mm×length 30 m×thickness 0.25 um)
Inlet temperature; 200° C.
Detector temperature; 280° C.

The obtained compound of Formula (7) was analyzed by ¹³C-NMR (100 MHz, CDCl₃), FT-IR, and MS.

¹³C-NMR (100 MHz, CDCl₃) δ 46.3, 72.5, 122.9, 127.8, 128.7, 129.1, 139.0, 140.3

FT-IR: 2260 cm⁻¹

MS: m/z=215(M′)

### (5) Content of 3-methylbenzyl isocyanate (Compound of Formula (4))

Methylbenzyl isocyanate (reagent, Sigma-aldrich) having a purity of 98% was used as a standard material and was carried out in the same manner as the measurement method of XDI to calculate the content of methylbenzyl isocyanate in the compositions of each Example and Comparative Example.

### (6) Refractive Index of Optical Lens

The refractive index (nₑ) of the optical lens obtained by the compositions of each Example and Comparative Example described below was measured at a wavelength of 546.1 nm (mercury e line) at 20° C. using a refractometer DR-M4 model manufactured by ATAGO.

### (7) Evaluation of Heat Resistance of Optical Lens

The glass transition temperature (Tg) was measured using a thermomechanical analysis device DSC N-650. The glass transition temperature was used as an index of heat resistance.

### (8) Yellow Index of the Optical Lens (Y.I.) and Light Transmittance

The Yellow Index of the optical lens was calculated by chromaticity coordinates x and y using UV-2600 240V EN (Shimadzu Corporation), and was expressed by Equation (1):

Y.I.=(234x+106y+106)/y [Equation 1]

### (9) Evaluation of Dyeing Properties (Colorability) of Optical Lens

Gray BPI #32000 (BP, dye) dye dispersion was prepared, and then a resin having a thickness of 2.0 mm was immersed in the dispersion in a water bath of 95° C. for 5 minutes to dye. The light transmittance (%) of the dyed resin was measured at the visible light total wavelength (380-780 nm).

Light is absorbed by the dye according to the degree of dyeing, and the lower the light transmittance at each wavelength, the better the dyeing property.

### (10) White Turbidity of Optical Lens

The white turbidity of the optical lens was evaluated with the naked eye based on the following criteria.

Good: transparent; Bad: white turbidity

### (11) Optical Lens Striae

The optical lens was visually observed under the mercury lamp, and when a non-uniform image was confirmed, it was classified as having a Striae.

The present invention is exemplified by the following examples, but is not limited thereto. First, a process of preparing XDI prepared under various conditions while changing reaction conditions in the preparation method of the present invention will be described.

### EXAMPLE

### 1. Synthesis of 1,3-Xylylene Diisocyanate (XDI) and Preparation of XDI Compositions Containing Chlorinated Derivatives

### Synthesis Example 1

After 7.3 kg of bis(trichloromethyl)carbonate was dissolved in 36 kg of o-dichlorobenzene in a 50 L reaction vessel equipped with a reflux cooler, a solution in which 2 kg of m-xylylenediamine was dissolved in 2 kg of o-dichlorobenzene, was slowly added thereto at 60° C. or less. The temperature was raised to 160° C., and then reacted for 4 hours while controlling the released hydrogen chloride gas. At the time of raising the temperature, a solution in which bis(trichloromethyl)carbonate was dissolved in o-dichlorobenzene, was further slowly added thereto, and stirring was smoothly performed. After completion of the reaction, nitrogen in the reactor was purged to remove unreacted phosgene and hydrogen chloride gas, and the obtained solution was filtered to obtain XDI containing chlorinated derivatives. The content of the chlorinated derivative in the XDI solution excluding o-dichlorobenzene was about 2.8%.

### Synthesis Example 2

The solution obtained in Synthesis Example 1 was desolvated, and pure XDI containing no chlorinated derivatives was obtained through a high vacuum distillation apparatus.

### Preparation Examples 1-3

As various isocyanate standard materials, the chlorinated derivatives (a mixture of the compounds of Chemical Formula (5), Chemical Formula (6), and Chemical Formula (7) in a predetermined ratio) was mixed with the XDI prepared in Synthesis Example 2, to obtain XDI compositions containing the chlorinated derivatives. Their amounts are 500 ppm (Preparation Example 1), 1400 ppm (Preparation Example 2), and 5000 ppm (Preparation Example 3), respectively, based on the total mass of the XDI solution.

### 2. Preparation of XDI Compositions Containing Non-Chlorinated Derivatives (Methylbenzyl Isocyanate) and/or Chlorinated Derivatives in the XDI Compositions Obtained in Preparation Examples 1 to 3

### (2-1) Examples 1 to 3

100 g of o-dichlorobenzene was added to 900 g of the XDI obtained in Preparation Examples 1 to 3, 2 g of Pd/C was added thereto, and then, the mixture was maintained while stirring under a 50° C., 2 kgf/cm² hydrogen atmosphere for 12 hours. After completion of the reaction, filtration under reduced pressure was performed, and desolvation and distillation process were performed to obtain an XDI composition containing methylbenzyl isocyanate.

### (2-2) Examples 4-10

Methylbenzyl isocyanate and a chlorinated derivative were mixed with the XDI obtained in Synthesis Example 1 with respect to the total mass of XDI to obtain an XDI composition containing all of them.

### (2-3) Example 11

0.2 g of Pd/C was added to 200 g of the XDI composition containing the actual chlorinated derivative obtained in Synthesis Example 1, and then the mixture was maintained while stirring under a 50° C., 2 kgf/cm² hydrogen atmosphere for 12 hours. After completion of the reaction, the reaction was filtered under reduced pressure and then desolvation process was performed to obtain XDI containing methylbenzyl isocyanate (2.7%) and a chlorinated derivative (0.06%). The obtained XDI solution was simply distilled under vacuum to obtain an XDI composition containing methylbenzyl isocyanate (0.5%) and chlorinated impurities (0.04%).

### (2-4) Comparative Examples 1 and 2

An XDI composition containing only chlorinated derivatives is obtained from the XDI obtained in Synthesis Example 2.

The contents of the methylbenzyl isocyanate and the chlorinated derivative are shown in Table 1 below, respectively, in the composition comprising methylbenzyl isocyanate in the XDI composition obtained by the reduction reaction in Examples 1 to 3, the XDI composition comprising the methylbenzyl isocyanate or the chlorinated derivative in Examples 4 to 10, the XDI composition comprising the methylbenzyl isocyanate and the chlorinated derivative by reducing the XDI comprising the chlorinated derivative prepared in the actual process in Example 11, and the XDI composition comprising only the chlorinated derivative in Comparative Examples 1 to 2.

TABLE 1

| | Contents of | Contents of |
|---|---|---|
| | methylbenzyl | chlorinated |
| Example | isocyanate (ppm) | derivative (ppm) |
| 1 | 600 | — |
| 2 | 1,600 | — |
| 3 | 5,600 | — |
| 4 | 0.05 | — |
| 5 | 0.1 | — |
| 6 | 30,000 | — |
| 7 | 30,000 | 900 |
| 8 | 50,000 | 1,500 |
| 9 | 50,000 | 1,600 |
| 10 | 60,000 | — |
| 11 | 5,210 | 430 |
| Com. | — | 1,600 |
| Exam. 1 | | |
| Com. | — | 30,000 |
| Exam. 2 | | |

### 3. Evaluation on the Conversion of Chlorinated Derivatives into Non-Chlorinated Derivatives by Reduction Reaction

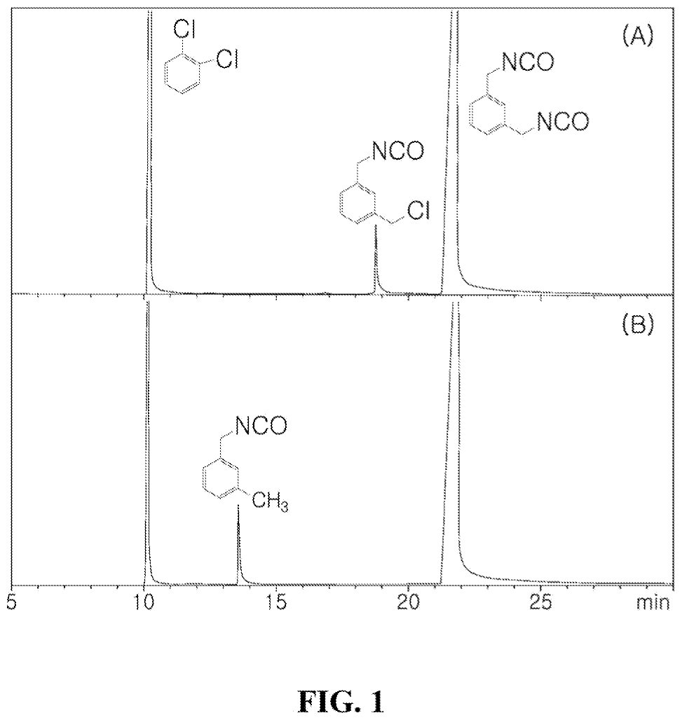

As shown in FIG. 1, this conversion process may be confirmed that the chlorinated derivatives before reduction reaction (A) are easily converted into non-chlorinated derivatives after reduction reaction (B) by a simple reduction process. In addition, although the difference in boiling points between XDI and chlorinated derivatives as impurities of XDI, was not large, separation and purification were difficult. However, the present invention confirmed that separation purification can be simplified by converting the chlorinated derivative into the non-chlorinated derivative having the large difference in boiling points.

### 4. Preparation and Evaluation of Optical Lenses from XDI Compositions

(4-1) Preparation of Optical Lens from XDI Composition of Examples 1 to 11 and Comparative Examples 1 to 2

52 g of the XDI composition prepared in Examples 1 to 11 and Comparative Examples 1 to 2, 0.015 g of dibutyltin dichloride, 0.12 g of Zerek UN (internal release agent, Stepan Co., Ltd.), and 0.08 g of ultraviolet absorber were mixed at room temperature for 1 hour, and 48 g of 2, 3-bis(2-mercaptoethylthio)propane-1-thiol was charged to prepare a polymerizable composition. The polymerizable composition was stirred under reduced pressure for 1 hour to remove bubbles, and was filtered through a 1 um Teflon filter. Then, the mixture was injected into a mold made of a glass mold and tape, and the temperature was gradually raised to 120° C. in an oven to be polymerized for 20 hours. The mold type was taken out from the oven and released to obtain a resin (plastic). The obtained resin was further annealed at 120° C. for 2 hours.

For each manufactured optical lens, physical properties were evaluated as follows and summarized in Table 2.

TABLE 2

| | Refractive | *Yellow | | | | | Heat |
|---|---|---|---|---|---|---|---|
| | index | index | **Chromaticity | ***Impact | White | | resistance |
| Example | (ne) | (Y.I) | (%) | resistance | turbidity | Striae | (Tg, ° C.) |
| 1 | 1.6656 | 1.22 | 68.1 | Good | Good | Good | 87.4 |
| 2 | 1.6655 | 1.23 | 67.5 | Good | Good | Good | 87.4 |
| 3 | 1.6654 | 1.32 | 67.1 | Good | Good | Good | 87.4 |
| 4 | 1.6656 | 1.15 | 69.9 | Good | Good | Good | 87.5 |
| 5 | 1.6656 | 1.20 | 68.5 | Good | Good | Good | 87.5 |
| 6 | 1.6646 | 1.26 | 66.0 | Good | Good | Good | 84.4 |
| 7 | 1.6645 | 1.30 | 66.6 | Good | Good | Good | 84.4 |
| 8 | 1.6645 | 1.38 | 66.1 | Good | Good | Good | 83.0 |
| 9 | 1.6644 | 1.40 | 66.4 | Good | Good | Good | 83.0 |
| 10 | 1.6644 | 1.65 | 65.9 | Bad | Good | Good | 81.2 |
| 11 | 1.6655 | 1.26 | 67.4 | Good | Good | Good | 87.2 |
| Com. | 1.6650 | 1.40 | 69.5 | Good | Good | Good | 87.4 |
| Exam. 1 | | | | | | | |
| Com. | 1.6646 | 2.30 | 71.2 | — | Bad | Bad | — |
| Exam, 2 | | | | | | | |

*The yellow index of the original lens (3 mm flat plate) obtained by the method of Examples 1 to 11 was measured.
**The light transmittance was measured by coloring the original lens obtained by the method of Examples 1 to 11 by the method for measuring the chromaticity.
***The impact resistance test was conducted in accordance with Drop Test (16.3 g * 127 cm).

(4-2) Evaluation of Physical Properties of Optical Lenses Prepared from XDI Compositions of Examples 1 to 11 and Comparative Examples 1 to 2
1) A lens using an XDI composition containing up to 50, 000 ppm of methylbenzyl isocyanate was usable as a super-refractive (nₑ20: 1.66 or more) lens.
2) When comparing Example 6 and Comparative Example 1, the yellow index was very different from 1.26 of Example 6 and 2.30 of Comparative Example 1, and a lens using an XDI composition containing methylbenzyl isocyanate in which a chlorinated derivative was reduced, showed excellent results in terms of yellow index compared with the chlorinated derivative. The XDI composition reduced to methylbenzyl isocyanate was superior in terms of yellowness compared with the XDI composition contained between chlorinated derivatives.
3) As shown in Examples 8 and 9 and Comparative Example 2, as the chlorinated derivative increased, the yellow index tended to increase. While the methylbenzyl isocyanate is the same at 50,000 ppm, a yellow index is 1.38 at 1,500 ppm of the chlorinated derivative, 1.40 at 1,600 ppm, 2.30 at 30,000 ppm, respectively, and the yellow index tends to increase as the chlorinated impurities increase. Therefore, when methylbenzyl isocyanate and the chlorinated derivative are contained together, the chlorinated derivative is preferably 1,500 ppm or less.
4) As shown in Comparative Examples 1 and 2 and Examples 3 and 10, as the chlorination impurities increase, the coloring efficiency decreases and the light transmittance of the colored lens increases, whereas as the amount of methylbenzyl isocyanates increases, the coloring efficiency increases and the light transmittance decreases, and thus unlike the chlorinated derivatives, methylbenzyl isocyanate contributes to the improvement of the coloring efficiency.
5) As shown in Example 6 and Comparative Example 2, the chlorinated derivative concentration (30, 000 ppm) resulted in defects in the white turbidity and the striae, but compared to the methylbenzyl isocyanate having the same concentration, it showed better results in the white turbidity and the striae, so that it could be used as an optical lens material.

1. A method for preparing an isocyanate composition comprising xylylene diisocyanate (XDI), comprising:
a step of converting a unreduced chlorinated derivative of Chemical Formula (1), into a reduced non-chlorinated derivative of Chemical Formula (4) by a reduction reaction,
wherein the prepared isocyanate composition comprises the reduced non-chlorinated derivative of Chemical Formula 4, together with the xylylene diisocyanate (XDI) and the unreduced chlorinated derivatives of Chemical Formula (1),
wherein a content ratio of the chlorinated derivative in the isocyanate composition is in the range of >0 to 1,500 ppm,
wherein a content ratio of the non-chlorinated derivative in the isocyanate composition is in the range of 0.1 to 50,000 ppm, 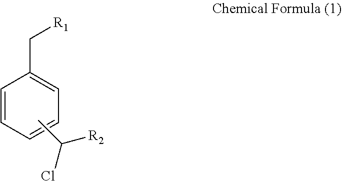
wherein, R1 is Cl or NCO, and R2 is H or Cl,

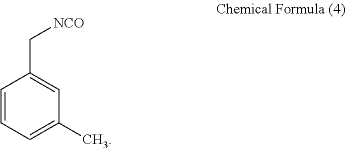

2. The method according to claim 1,
wherein the xylylene diisocyanate (XDI) is obtained by directly reacting an organic primary amine with a carbonylating agent under an inert solvent according to the following reaction scheme:

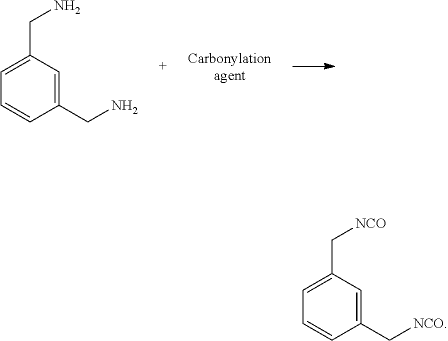

3. The method according to claim 1,
wherein the above xylylene diisocyanate (XDI) is obtained by (i) reacting an organic primary amine with hydrogen chloride gas to obtain an amine hydrochloride salt and (ii) reacting the amine hydrochloride salt obtained from the first step with a carbonylating agent, according to the following reaction scheme:
Step 1 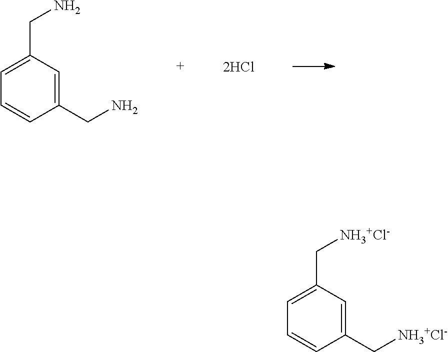
Step 2

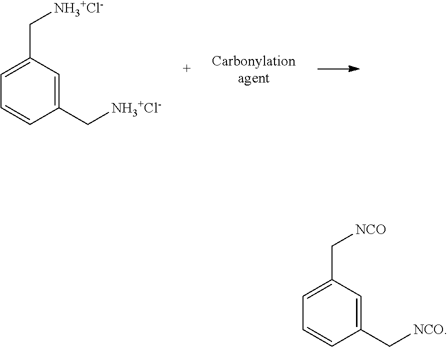

4. The method according to claim 2,
wherein the carbonylating agent is selected from the group consisting of phosgene, diphosgene, triphosgene, and a combination thereof.

5-9. (canceled)

10. An isocyanate composition comprising:
xylylene diisocyanate (XDI);
a chlorinated derivative of Chemical Formula (1); and
a non-chlorinated derivative of Chemical Formula (4),
wherein the chlorinated derivative of Chemical Formula (1) is an unreduced chlorinated derivative generated during the preparing process of the XDI and the non-chlorinated derivative of Chemical Formula 4 is obtained from the unreduced chlorinated derivative by a reduction reaction,
wherein a content ratio of the chlorinated derivative in the isocyanate composition is in the range of >0 to 1,5001 ppm,
wherein a content ratio of the non-chlorinated derivative in the isocyanate composition is in the range of 0.1 to 50,000 ppm, 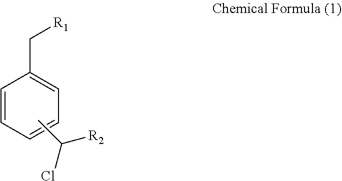
wherein, R1 is Cl or NCO, and R2 is H or Cl.

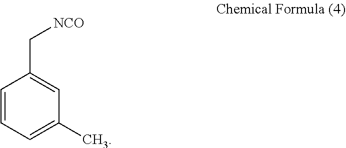

11. (canceled)

12. The isocyanate composition according to 10,
wherein the unreduced chlorinated derivative is a chlorinated derivative represented by Chemical Formula (1) and is at least one of chloromethyl benzyl isocyanate, xylylene dichloride, dichloromethyl benzylisocyanate, and chloromethyl dichloromethyl benzene, 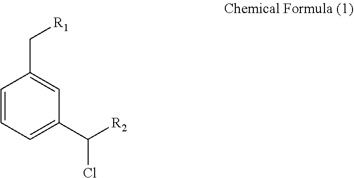
R1 is Cl or NCO and R2 is H or Cl.

13. The isocyanate composition according to claim 10,
wherein a content ratio of the unreduced chlorinated derivative is in the range from >0 to 900 ppm.

14. The isocyanate composition according to claim 10,
wherein a content ratio of xylylene diisocyanate in the composition is 99 mass percent or more.

15. A polymerizable composition, comprising:
the isocyanate composition according to claim 10; and
a compound containing at least one isocyanate-reactive functional group.

16. The polymerizable composition according to claim 15,
wherein the compound containing at least one isocyanate-reactive functional group is a polyol compound or a polythiol compound.

17. A polymerizable composition, comprising:
the isocyanate composition according to claim 11; and
a compound containing at least one isocyanate-reactive functional group.

18. The polymerizable composition according to claim 17,
wherein the compound containing at least one isocyanate-reactive functional group is a polyol compound or a polythiol compound.

19. The polymerizable composition according to claim 16,
wherein the polymerizable composition is used as an optical resin after polymerization reaction.

20. The polymerizable composition according to claim 18,
wherein the polymerizable composition is used as an optical resin after polymerization reaction.

21-24. (canceled)

25. The method according to claim 3,
wherein the carbonylating agent is selected from the group consisting of phosgene, diphosgene, triphosgene, and a combination thereof.

26. The method according to claim 1,
wherein the reduction reaction is performed using a metal catalyst or an reducing agent under a hydrogen (H2) atmosphere.

27. The method according to claim 26,
wherein the metal catalyst is at least one of palladium, platinum and nickel.

28. The method according to claim 26,
wherein the reducing agent is at least one selected from the group consisting of following: lithium aluminum hydride (LiAlH4), sodium amalgam (Na(Hg)), zinc amalgam (Zn(Hg)), diboane (B2H6), lithium borohydride (LiBH4), sodium borohydride (NaBH4), iron(II) sulfate (FeSO4), tin chloride(II) (SnCl2), sodium dithionate (Na2S2O6), sodium thiosulfate (Na2S2O3), ammonium thiosulfate (NH4)2S2O3), diisobutylaluminum hydride (DIBAL-H), oxalic acid (C2H2O4), formic acid (HCOOH), dithiothreitol (DTT), and dithiothreitol (DTT), tris-2-carboxyethylphosphine hydrochloride (TCEP), 2,2-diphenyl-1-picrylhydrazyl (DPPH), butylated hydroxytoluene (BHT), 2,6-di-tert-butyl-4-methylphenol, 2,4 dimethyl-6-tert-butylphenol, trialkyltin hydride, tributyltin hydride, and zinc.

29. The method according to claim 1,
wherein separation and purification process are performed by simple distillation after the reduction reaction.

30. The isocyanate composition according to claim 10,
wherein the xylylene diisocyanate is obtained by directly reacting an organic primary amine with a carbonylating agent in an inert solvent according to the following reaction scheme: [chemical structure]
